(19) United States
(12) Patent Application Publication
(10) Pub. No.: US 2024/0041743 A1
(43) Pub. Date: Feb. 8, 2024

(54) SYNERGISTIC TETRAPEPTIDE COMBINATION

(71) Applicants: BASF CORPORATION, Florham Park, NJ (US); BASF Beauty Care Solutions France SAS, Lyon (FR)

(72) Inventors: Manasi Chavan, Nanuet, NY (US); Philip Ludwig, Dunellen, NJ (US); Jill Litchauer, Brewster, NY (US); Clifford Milow, Massapequa, NY (US); Juan Brito, Garnerville, NY (US); Philippe Moussou, Tomblaine (FR)

(21) Appl. No.: 18/326,183

(22) Filed: May 31, 2023

Related U.S. Application Data
(62) Division of application No. 16/220,066, filed on Dec. 14, 2018, now abandoned.
(60) Provisional application No. 62/611,611, filed on Dec. 29, 2017.

(51) Int. Cl. A61K 8/64; A61K 8/34; A61K 8/37; A61K 8/44; A61K 8/49; A61K 8/60; A61K 8/73; A61K 8/81; A61K 8/86; A61Q 19/08; A61K 38/07

(52) U.S. Cl. CPC A61K 8/64; A61K 8/345; A61K 8/375; A61K 8/44; A61K 8/4973; A61K 8/602; A61K 8/73; A61K 8/8111; A61K 8/8147; A61K 8/86; A61Q 19/08; A61K 38/07

(57) ABSTRACT

The present invention discloses a cosmetic composition for the treatment of forehead wrinkles, nose fold, crow's feet, cheek density, and cheek hypoechogenic dermal factor.

### FIELD OF THE INVENTION

The present invention relates to cosmetic compositions for skin care and anti-aging applications. Particularly to a cosmetic composition for skin firmness and its methods of use.

### SEQUENCE LISTING

The instant application contains a Sequence Listing which has been filed electronically in XML format and is hereby incorporated by reference in its entirety. Said XML copy, created on Sep. 6, 2023, is named 31559-590_SL-generated-by-WIPO.XML and is 3,960 bytes in size.

### BACKGROUND OF THE INVENTION

Nowadays, individuals often seek means for counteracting aging effects. A variety of cosmetic compositions as well as homeopathic remedies are available to the consumer looking obtain a more youthful image. As a result, consumer's expectations are ever increasing with consumers expecting fast acting products, natural products, products that simultaneously provide a plurality of benefits (e.g., storage stability, photostability, constitute active ingredients which confer care, irritation-suppressing and/or photoprotective properties etc).

The object of the present patent application is to provide cosmetic compositions which ma be used to prevent or treat signs of skin ageing that may be expressed as loss of firmness, decrease of skin thickness, fine lines, wrinkles, loss of elasticity, sagging, dryness, age spots, diminished rate of turnover, abnormal desquamation, decrease of the density and disorganization of the extra-cellular matrix in the dermis and other histological changes.

### SUMMARY

It is an object of certain embodiments of the disclosure to provide a cosmetic composition for improving the appearance of the skin, skin elasticity, body firming or to reduce the visible signs of ageing.

It is a further object of certain embodiments of the disclosure to provide a cosmetic composition for treating forehead wrinkles.

It is a further object of certain embodiments of the disclosure to provide a cosmetic composition for treating nasal folds.

It is a further object of certain embodiments of the disclosure to provide a cosmetic composition for treating crow's feet.

It is a further object of certain embodiments of the disclosure to provide a cosmetic composition for increasing cheek density.

It is a further object of certain embodiments of the disclosure to provide a cosmetic composition for improving hypoechogenic dermal factor.

The above objects of the present invention and others may be achieved by the present invention which in certain embodiments is directed to a cosmetic composition comprising a synergistic combination of (i) N-Acetyl-Glutaminyl-Aspartyl-Valyl-Histidine (SEQ ID NO:1), and (ii) N-Acetyl-Prolyl-Prolyl-Tyrosyl-Leucine (SEQ ID NO:2).

In some embodiments, the present invention is directed to a cosmetic composition consisting essentially of a combination of (i) N-Acetyl-Glutaminyl-Aspartyl-Valyl-Histidine (SEQ ID NO:1); (ii) N-Acetyl-Prolyl-Prolyl-Tyrosyl-Leucine (SEQ ID NO:2); and at least one cosmetically acceptable excipient.

In some embodiments, the present invention is directed to a cosmetic composition comprising a combination of (i) N-Acetyl-Glutaminyl-Aspartyl-Valyl-Histidine (SEQ ID NO:1), and (ii) N-Acetyl-Prolyl-Prolyl-Tyrosyl-Leucine (SEQ ID NO:2), wherein the molar ratio of SEQ ID NO:1 to SEQ ID NO:2 in the cosmetic composition is about 1.3:1 to about 1:2.3, about 1.2:1 to about 1:2.1, about 1.1:1 to about 1:1.6, or about 1:1.3.

In some embodiments, the present invention is directed to a method of treating forehead wrinkles comprising topically administering a cosmetic composition according to an embodiment to the forehead of a subject in need thereof.

In some embodiments, the present invention is directed to a method of treating nasal folds comprising topically administering a cosmetic composition according to an embodiment to the affected area of a subject in need thereof.

In some embodiments, the present invention is directed to a method of treating crow's feet comprising topically administering a cosmetic composition according to an embodiment to the affected area of a subject in need thereof.

In some embodiments, the present invention is directed to a method of increasing cheek density comprising topically administering a cosmetic composition according to an embodiment to the cheek of a subject in need thereof.

In some embodiments, the present invention is directed to a method of improving hypoechogenic dermal factor comprising topically administering a cosmetic composition according to an embodiment to the affected area of a subject in need thereof.

The term “synergistic combination” as used herein means that the combined effect of SEQ ID NO:1 and SEQ ID NO:2 is greater than the sum of their separate effects with respect to the exhibited improvement in one or more of forehead wrinkles, nasal fold, crow's feet, cheek density, or hypoechogenic dermal factor after at least a 7 day treatment, at least a 10 day treatment, at least a 21 day treatment, at least a 42 day treatment, or at least a 56 day treatment.

The term “epidermal penetration enhancer” as used herein refers to an agent that increases the rate at which an active agent will pass through the skin barrier and get absorbed into the skin.

The term “administering the cosmetic composition” as used herein refers to applying topically onto an affected area SEQ ID NO:1 and SEQ ID NO:2 either sequentially as two separate cosmetic compositions (each separate cosmetic composition comprising either SEQ ID NO:1 or SEQ ID NO:2), concurrently as two separate cosmetic compositions (each separate cosmetic composition comprising either SEQ ID NO:1 or SEQ ID NO:2), or concurrently as a single cosmetic composition that comprises both SEQ ID NO:1 and SEQ ID NO:2. If SEQ ID NO:1 and SEQ ID NO:2 are applied sequentially, they may be applied to the affected area in any order.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIGS. 1A-1C illustrate the improvement in forehead wrinkles, nasal fold and crow's feet, respectively, after 21 and 56 day treatment of the affected area with a peptide combination according to an embodiment.

FIG. 2 illustrates the improvement in cheek dermal density, as measured by ultrasound, after 21 and 56 days treatment of the affected area with a peptide combination according to an embodiment.

FIG. 3 illustrates the improvement in cheek hypoechogenic dermal factor, as measured by ultrasound, after 21 and 56 days treatment of the affected area with a peptide combination according to an embodiment.

### DETAILED DESCRIPTION

### Cosmetic Composition

According to various embodiments, the present disclosure is related to a cosmetic composition comprising a synergistic combination of (i) N-Acetyl-Glutaminyl-Aspartyl-Valyl-Histidine (SEQ ID NO:1), and (ii) N-Acetyl-Prolyl-Prolyl-Tyrosyl-Leucine (SEQ ID NO:2).

In some embodiments, the present invention is directed to a cosmetic composition consisting essentially of a combination of (i) N-Acetyl-Glutaminyl-Aspartyl-Valyl-Histidine (SEQ ID NO:1); (ii) N-Acetyl-Prolyl-Prolyl-Tyrosyl-Leucine (SEQ ID NO:2); and at least one cosmetically acceptable excipient.

The amino acids can either occur in the L, the D, or the DL form in SEQ ID NO:1 and in SEQ ID NO:2. In some embodiments, the amino acids are all in L form.

SEQ ID NO:1 is also known as Acetyl Tetrapeptide-9 and is commercially available as Dermican™ by BASF Care Creation. It is a pure tetrapeptide having an optimal collagen fibers functionality via a specific anchoring small proteoglycan Lumican. It is believed that Dermican™ increases lumican levels in aged skin fibroblasts.

SEQ ID NO:2 is also known as Acetyl Tetrapeptide-11 and is commercially available as Syniorage™ by BASF Care Creation. This tetrapeptide stimulates Syndecan expression and production of Collagen 17 in skin keratinocytes.

The combination of SEQ ID NO:1 and of SEQ ID NO:2 is especially useful for producing cosmetic compositions, which can be used to improve the appearance of the skin, body firming or reduce the visible signs of ageing. In particular these cosmetic compositions are used to prevent and/or to diminish intrinsic and/or extrinsic skin ageing, to delay the outcome of wrinkles, to reduce the depth of installed wrinkles, to diminish the appearance of fine lines, to improve the skin roughness, to prevent and/or to diminish skin sagging, to improve biomechanical properties e.g. such as skin firmness, skin elasticity, to prevent and/or to diminish the appearance of age spots, to improve the skin's regenerative and renewal process, to help rejuvenation of the aged or stressed human skin. The topical cosmetic compositions containing the peptide combination according to the invention may be also used to help skin to fight against external or environmental stresses, such as oxidative stress, UV rays, irritants, allergens, pollution and various environmental toxins.

In some embodiments, the cosmetic composition comprises a molar ratio of SEQ ID NO: 1 to SEQ ID NO:2 ranging from about 2:1 to about 1:10, from about 1.5:1 to about 1:8, from about 1.3:1 to about 1:6, from about 1.3:1 to about 1:4, from about 1.3:1 to about 1:2.5, from about 1.3:1 to about 1:2.3, from about 1.2:1 to about 1:2.1, from about 1.1:1 to about 1:1.6, from about 1.2:1 to about 1:1.6, or about 1:1.3.

In some embodiments, the cosmetic composition comprises from about 5 ppm to about 1200 ppm, from about 10 ppm to about 1200 ppm, from about 15 ppm to about 1200 ppm, from about 20 ppm to about 1200 ppm, from about 30 ppm to about 1200 ppm, from about 50 ppm to about 1200 ppm, from about 100 ppm to about 1200 ppm, from about 250 ppm to about 1200 ppm, from about 500 ppm to about 1200 ppm, from about 750 ppm to about 1200 ppm, from about 900 ppm to about 1200 ppm, from about 1000 ppm to about 1100 ppm of SEQ ID NO:1 or of SEQ ID NO:2, independently.

When the term “cosmetic composition” refers to a concentrated composition that may act as a cosmetic ingredient incorporated into a variety of formulations to form a final consumer commercial product, the concentrations may range from about 250 to about 450 ppm of SEQ ID NO:1 and from about 350 ppm to about 550 ppm of SEQ ID NO:2, from about 400 ppm to about 650 ppm of SEQ ID NO:1 and from about 500 ppm to about 850 ppm of SEQ ID NO:2, or from about 800 ppm to about 1300 ppm of SEQ ID NO:1 and from about 1050 ppm to about 1650 ppm of SEQ ID NO:2.

When the term “cosmetic composition” refers to a final consumer commercial product, the concentrations of SEQ ID NO:1 and SEQ ID NO:2 described herein may be diluted. For instance, in a final consumer product, SEQ ID NO:1 may be present at a concentration ranging from about 5 ppm to about 15 ppm, or from about 8 ppm to about 13 ppm and SEQ ID NO:2 may be present at a concentration ranging from about 5 ppm to about 20 ppm, or from about 10 ppm to about 17 ppm.

The cosmetic compositions according to embodiments exhibit an improvement in one or more of forehead wrinkles, nasal fold, crow's feet, cheek density, or hypoechogenic dermal factor after a 10 day treatment, after a 14 day treatment, after a 21 day treatment, after a 42 day treatment, or after a 56 day treatment.

In some embodiments, the cosmetic composition comprises at least one cosmetically acceptable excipient. In some embodiments, the at least one cosmetically acceptable excipient may be present in the cosmetic composition at a concentration ranging from about 80 wt % to about 99.5 wt %, from about 90 wt % to about 99 wt %, from about 95 wt % to about 98.5 wt %, from about 96 wt % to about 98 wt %, from about 20 wt % to about 60 wt %, from about 30 wt % to about 55 wt %, or from about 45 wt % to about 55 wt %, based on total weight of the cosmetic composition.

The cosmetically acceptable excipient may comprise an epidermal penetration enhancer, a solvent, mild surfactants, oil bodies, emulsifiers, pearlescent waxes, consistency regulators, thickeners, superfatting agents, stabilizers, polymers, silicone compounds, fats, waxes, lecithins, phospholipids, UV photoprotective factors, biogenic active ingredients, antioxidants, deodorants, antiperspirants, antidandruff agents, film formers, swelling agents, insect repellents, self-tanning agents, tyrosinase inhibitors, hydrotropes, solubilizers, preservatives, perfume oils, dyes, or combinations thereof.

In certain embodiments, the cosmetic composition comprises a cosmetically acceptable excipient comprising an epidermal penetration enhancer. Exemplary epidermal penetration enhancers may include, but not be limited to, monohydric alcohols, polyhydric alcohols, saturated and unsaturated fatty alcohols having 8 to 10 carbon atoms, hydrocarbons, saturated and unsaturated fatty acids, fatty acid esters having up to 24 carbon atoms, dicarboxylic acid diesters with up to 24 carbon atoms, phosphate derivatives, ethers, polyethoxyethylene, EDTA, nerolidol, lime oxide, phospholipids, SDS (sodium dodecyl sulfate), dimethylformamide, N-methyl formamide, hydrophobins and combinations thereof.

In some embodiments, the monohydric alcohols and polyhydric alcohols comprise ethanol, 1,2-propanediol, benzyl alcohol, or combinations thereof. In some embodiments, the saturated and unsaturated fatty alcohols having 8 to 10 carbon atoms comprise lauryl alcohol, cetyl alcohol, or combinations thereof. In some embodiments, the hydrocarbons comprise mineral oil, alkanes, esters, azone, propylene glycol, chitosan, or combinations thereof. In some embodiments, the saturated and unsaturated fatty acids comprise stearic acid, oleic acid, or combinations thereof. In some embodiments, the fatty acid esters having up to 24 carbon atoms or dicarboxylic acid diesters with up to 24 carbon atoms comprise methyl ester, ethyl ester, isopropyl ester, butyl ester, sec-butyl ester, isobutyl ester, tert butyl ester, monoglycerin esters of acetic acid, caproic acid, lauric acid, myristic acid, stearic acid, palmitic acid, or combinations thereof. In some embodiments, the phosphate derivatives comprise lecithin, terpenes, urea, urea derivatives, or combinations thereof. In some embodiments, ethers comprise dimethyl isosorbide, diethylene glycol monoethyl ether, or combinations thereof. In one embodiment, the epidermal penetration enhancer comprises dimethyl isosorbide.

In some embodiments, the epidermal penetration enhancer may be present in the cosmetic composition at a concentration ranging from about 0.5 wt % to about 95 wt %, from about 1 wt % to about 90 wt %, from about 1.5 wt % to about 70 wt %, from about 2 wt % to about 50 wt %, from about 2 wt % to about 40 wt %, from about 2 wt % to about 30 wt %, from about 2 wt % to about 25 wt %, from about 0.5 wt % to about 5 wt %, from about 1 wt % to about 3 wt %, from about 1 wt % to about 2 wt %, from about 40 wt % to about 70 wt %, from about 45 wt % to about 60 wt %, or from about 48 wt % to about 52 wt %, based on total weight of the cosmetic composition.

In certain embodiments, the cosmetic composition comprises a cosmetically acceptable excipient comprising a solvent. For instance, the combination of SEQ ID NO:1 and SEQ ID NO:2 may be diluted in a cosmetically acceptable solvent. Exemplary solvents include, but are not limited to, polysorbate 20, water, propylene glycol, butylene glycol, ethoxylated or propoxylated diglycol, ethanol, propanol, isopropanol glycerol, or mixtures thereof. In one embodiment, the solvent comprises polysorbate 20.

In some embodiments, the solvent may be present in the cosmetic composition at a concentration ranging from about 0.5 wt % to about 95 wt %, from about 1 wt % to about 90 wt %, from about 1.5 wt % to about 70 wt %, from about 2 wt % to about 50 wt %, from about 2 wt % to about 40 wt %, from about 2 wt % to about 30 wt %, from about 2 wt % to about 25 wt %, from about 0.5 wt % to about 5 wt %, from about 1 wt % to about 3 wt %, from about 1 wt % to about 2 wt %, from about 40 wt % to about 70 wt %, from about 45 wt % to about 60 wt %, or from about 48 wt % to about 52 wt %, based on total weight of the cosmetic composition.

In certain embodiments, the cosmetic composition comprises a cosmetically acceptable excipient comprising an epidermal penetration enhancer and a solvent. In certain embodiments, the cosmetic composition comprises dimethyl isosorbide and polysorbate 20.

Cosmetic, Pharmaceutical and/or Dermatological Preparations

The uses of the present invention may include the production of cosmetic or pharmaceutical preparations, such as, for example, shampoos, hair lotions, bath foams, bath and shower preparations, cremes, gels, lotions, alcohol or aqueous/alcohol solutions, emulsions, fatty/waxy material substances, stick type preparations, powders or ointments. Those preparations may further contain, as admixtures or additives, mild surfactants, fatty bodies, emulsifying agents, pearl-luster waxes, consistency agents, thickening agents, super-fatty agents, stabilizing agents, polymers, silicone-containing compounds, fatty substances, waxes, lecithin, phospholipids, factors for protection against UV rays, biogenic substances, antioxidants, deodorants, antiperspirants, anti-film agents, film-forming agents, expansion agents, insect repellents, self-tanning agents, tyrosine inhibitors (depigmentation agents), hydrotropes, solubilizers, preserving agents, perfume oils and dyes, or the like.

### Surfactants

Anionic, non-ionic, cationic and/or amphoteric or zwitterionic surfactants may be contained as surface-active substances, and their proportion relative to the media is conventionally from 1 to 70, preferably from 5 to 50, and in particular from 10 to 30% by weight. Typical examples of anionic surfactants are soaps, alkylbenzene sulphonates, alkane sulphonates, olefin sulphonates, alkylether sulphonates, glycerin ether sulphonates, α-methylester sulphonates, sulphonated fatty acids, alkyl sulphates, alkylether sulphates, glycerin ether sulphates, fatty acid ether sulphates, mixed ether/hydroxyl sulphates, monoglycerin ether sulphates, fatty acid amide ether sulphates, mono- and dialkylsulphonates, mono- and dialkylsulphosuccinamates, sulphoglycerides, amide soaps, ether carbonic acids and the salts thereof, fatty acid isethionates, fatty acid sarcocinates, fatty acid taurides, N-acyl amino acids, such as, for example, acyl lactilate, acyl tartrate, acyl glutamate and acyl asparate, alkyloligoglucoside sulphate, protein-carrying fatty acid condensates (in particular, vegetable wheat-based products) and alkyl(ether) phosphates. Typical examples of non-ionic surfactants are fatty alcohol polyglycol ether, alkyl phenol polyglycol ether, fatty acid polyglycol ester, fatty acid amide polyglycol ether, fatty amino polyglycol ether, alkoxylated triglycerides, mixed ethers or mixed formals, optionally partially oxidised alkylenyl oligoglycosides or derivatives of glucoronic acid, fatty acid N-alkyl glucamides, protein hydrolysates (in particular, vegetable wheat-based products), polyol fatty acid esters, sugar esters, sorbitan esters, polysorbate esters and amino oxide esters. Examples of cationic surfactants include quaternary ammonium compounds, such as aluminum chloride of dimethyldistearyl, the esterquats, salts of ester of quaternised fatty acid trialkanolamine. Examples of amphoteric or zwitterionic surfactants include alkylbetaine, alkylamidobetaine, aminopropionate, aminoglycinate, imidazoliniumbetaine and sulphobetaine. Examples of suitable mild surfactants, that is to say, those which are particularly well-tolerated by the skin, are fatty alcohol polyglycol ether sulphates, monoglyceride sulphates or mono- and/or dialkyl sulphosuccinates, fatty acid isethionates, fatty acid sarcocinates, fatty acid taurides, fatty acid glutamates, α-olefin sulphonates, ether carbonic acids, alkyl oligoglucosides, fatty acid glucamides, alkyl amidobetaines, protein fatty acid amphoacetals and/or condensates.

### Fatty Bodies

There are considered, as fatty bodies, for example, Guerbet alcohols based on a fatty alcohol having from 6 to 18, preferably 8 to 10 carbon atoms, linear C6-C22 fatty acid esters with linear or branched C6-C22 fatty alcohols or C6-C13 carbonic acid esters which are branched with linear or branched C6-C22 fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. In the same manner, linear C6-C22 fatty acid esters with branched alcohols may be suitable, for example, 2-ethylhexanol, C18-C38 alkyl hydroxycarboxylic acid ester with linear or branched C6-C22 alcohols, for example, dioctyl malate, linear and/or branched fatty acid esters with polyfunctional alcohols (such as, for example, propylene glycol, diol dimer or triol trimer) and/or Guerbet alcohols, triglycerides based on C6-C10 fatty acids, mixtures of mono-/di- or triglycerides based on C6-C18 fatty acids, C6-C22 fatty alcohol esters and/or Guerbet alcohols with aromatic carbonic acids, such as, benzoic acids, C2-C12 dicarbonic acid esters with linear or branched alcohols having from 1 to 22 carbon atoms or polyols having from 2 to 10 carbon atoms and from 2 to 6 hydroxyl groups, vegetable oils, branched primary alcohols, substituted cyclohexanes, linear or branched C6-C22 fatty alcohol carbonates, such as dicaprylyl carbonate (Cetiol® CC), Guerbet carbonate based on fatty alcohols having from 6 to 18 or from 8 to 10 carbon atoms, benzoic acid esters with linear and/or branched C6-C22 alcohols (for example, Finsolv® TN), linear or branched, symmetrical or non-symmetrical dialkyl ethers having from 6 to 22 carbon atoms per alkyl group, such as, for example, dicaprylyl ether (Cétiol® OE), ring opening products of fatty acid esters which are epoxydised with polyols, silicone oil (cyclomethicone, types of silicon methicone, inter alia) and/or aliphatic or naphthenic hydrocarbons, such as, for example, squalane, squalene or dialkyl cyclohexane.

### Emulsifying Agents

Suitable emulsifiers may be, for example, non-ionogenic surfactants from at least one of the following groups:
addition products of from 2 to 30 mol of ethylene oxide and/or 0 to 5 mol of propylene oxide to linear fatty alcohols having 8 to 22 carbon atoms, to fatty acids having 12 to 22 carbon atoms, to alkylphenols having 8 to 15 carbon atoms in the alkyl group, and alkylamines having 8 to 22 carbon atoms in the alkyl radical;
alkyl and/or alkenyl oligoglycosides having 8 to 22 carbon atoms in the alk(en)yl radical and the ethoxylated analogues thereof
addition products of from 1 to 15 mol of ethylene oxide to castor oil and/or hydrogenated castor oil;
addition products of from 15 to 60 mol of ethylene oxide to castor oil and/or hydrogenated castor oil;
partial esters of glycerol and/or sorbitan with unsaturated, linear or saturated, branched fatty acids having 12 to 22 carbon atoms d/or hydroxycarboxylic acids having 3 to 18 carbon atoms, and the adducts thereof with 1 to 30 mol of ethylene oxide;
partial esters of polyglycerol (average degree of self-condensation 2 to 8), polyethylene glycol (molecular weight 400 to 5 000), trimethylolpropane, pentaerythritol, sugar alcohols (e.g. sorbitol), alkyl glucosides (e.g. methyl glucoside, butyl glucoside, lauryl glucoside), and polyglucosides cellulose with saturated and/or unsaturated, linear or branched fatty acids having 12 to 22 carbon atoms and/or hydroxycarboxylic acids having 3 to 18 carbon atoms, and the adducts thereof with 1 to 30 mol of ethylene oxide;
mixed esters of pentaerythritol, fatty acids, citric acid and fatty alcohol and/or mixed esters of fatty acids having 6 to 22 carbon atoms, methylglucose and polyols, preferably glycerol or polyglycerol,
mono-, di- and trialkyl phosphates, and mono-, di- and/or tri-PEG alkyl phosphates and salts thereof;
wool wax alcohols;
polysiloxane-polyalkyl-polyether copolymers and corresponding derivatives;
block copolymer, e.g. polyethylene glycol-30 dipolyhydroxystearates;
polymer emulsifiers. e.g. Pemulen grades (TR-1, TR-2) from Goodrich;
polyalkylene glycols, and
glycerol carbonate.

### Ethylene Oxide Addition Products

The addition products of ethylene oxide and/or of propylene oxide to fatty alcohols, fatty acids, alkylphenols or to castor oil are known, commercially available products. These are homologue mixtures whose average degree of alkoxylation corresponds to the ratio of the amounts of substance of ethylene oxide and/or propylene oxide and substrate with which the addition reaction is carried out. C12/18-fatty acid mono- and diesters of addition products of ethylene oxide to glycerol are known as refatting agents for cosmetic preparations.

Alkyl and/or Alkenyloligoglycoside

Alkyl and alkenyloligoglycoside, production thereof and use thereof are known according to the prior art. Its production is carried out in particular by conversion of glucose or oligosaccharides with primary alcohols with 8 to 18 carbon atoms. With regard to the residues of glycosides, it is considered that both monoglucosides to which a residue of cyclical sugar is bonded by means of a glycoside at the fatty alcohol and oligomeric glycosides with a degree of oligomerisation which can preferably be up to 8 are suitable. Consequently, the degree of oligomerisation is a statistical intermediate value, on which a conventional homogeneous distribution is based for those technical products.

### Partial Glycerides

Typical examples of suitable partial glycerides are hydroxystearic acid monoglyceride, hydroxystearic acid diglyceride, isostearic acid monoglyceride, isostearic acid diglyceride, olefinic acid monoglyceride, olefinic acid diglyceride, ricinolic acid monoglyceride, ricinolic acid diglyceride, linolic acid monoglyceride, linolic acid diglyceride, linolenic acid monoglyceride, linolenic acid diglyceride, erucic acid monoglyceride, erucic acid diglyceride, tartric acid monoglyceride, tartric acid diglyceride, citric acid monoglyceride, citric acid diglyceride, maleic acid monoglyceride, maleic acid diglyceride as well as the technical admixtures thereof which may further contain minute quantities of subordinate triglycerides in the production process. Addition products of from 1 to 30, preferably 5 to 10 moles of ethylene oxide relative to the partial glycerins cited are also suitable.

### Sorbitan Esters

There are considered, as the sorbitan ester, sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitane trihydroxystearate, sorbitan monotartrate, sorbitan sesquitartrate, sorbitan ditartrate, sorbitan tritartrate, sorbitan monocitrate, sorbitan sesquicitrate, sorbitan dicitrate sorbitan tricitrate, sorbitan monomaleate, sorbitan sesquimaleate, sorbitan dimaleate, sorbitan trimaleate as well as the technical admixtures thereof. Suitable addition products also include those having from 1 to 30, preferably from 5 to 10 moles of ethylene oxide in the sorbitan ester mentioned.

### Polyglycerin Esters

Typical examples of suitable polyglycerin esters are polyglyceryl-2 dipolyhydroxystearate (Dehymuls® PGPH), polyglycerin-3-diisostearate (Lameform® TGI), polyglyceryl-4 isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (Polyglycerol Caprate T2010/90), polyglyceryl-3 cethyl ether (Chimexane® NL), polyglyceryl-3 distearate (Cremophor® GS 32) and polyglyceryl polyricinoleate (Admul® WOL 1403), polyglyceryl dimerate isostearate, as well as admixtures thereof. Examples of polyol ester which are also suitable are the mono-, di- and triesters of trimethylolpropane or pentaerythritol which may be optionally reacted with 1 to 30 moles of ethylene oxide, of trimethylolpropane or pentaerythritol with lauric acid, fatty coconut acid, fatty tallow acid, palm acid, stearic acid, oleic acid, behenic acid and the like.

### Anionic Emulsifying Agents

Typical anionic emulsifying agents are fatty aliphatic acids having from 12 to 22 carbon atoms, such as, for example, palm acid, stearic acid, behenic acid as well as dicarbonic acids having from 12 to 22 carbon atoms, such as, for example, azelaic acid or sebacic acid.

### Amphoteric and Cationic Emulsifying Agents

Furthermore, zwitterionic surfactants can be used as emulsifiers. The term “zwitterionic surfactants” refers to those surface-active compounds which carry at least one quaternary ammonium group and at least one carboxylate and one sulphonate group in the molecule. Suitable zwitterionic surfactants are the so-called betaines, such as N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethylhydroxyethylcarboxymethyl glycinate. Another suitable example is a fatty acid amide derivative known under the CTFA name Cocamidopropyl Betaine. Likewise suitable emulsifiers are ampholytic surfactants. The term “ampholytic surfactants” means those surface-active compounds which, apart from a C8/18-alkyl or -acyl group, contain at least one free amino group and at least one —COON or —SO3H group in the molecule and are capable of forming internal salts. Examples of suitable ampholytic surfactants are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Other suitable ampholytic surfactants include N-cocoalkylaminopropionate, cocoacylaminoethylaminopropionate and C12/18-acylsarcosine. Cationic surfactants are also suitable as emulsifiers, those of the ester quat type, such as methyl-quaternized difatty acid triethanolamine ester salts.

### Fatty Substances and Waxes

Typical examples of a fatty substance are glycerides, i.e. solid or liquid vegetable or animal products which consist essentially of mixed glycerol esters of higher fatty acids, suitable waxes are inter alia natural waxes, such as, for example, candelilla wax, carnauba wax, Japan wax, esparto grass wax, cork wax, guaruma wax, rice germ oil wax, sugarcane wax, ouricury wax, montan wax, beeswax, shellac wax, spermaceti, lanolin (wool wax), uropygial grease, ceresin, ozokerite (earth wax), petrolatum, paraffin waxes, microcrystalline waxes; chemically modified waxes (hard waxes), such as, for example, montan ester waxes, sasol waxes, hydrogenated jojoba waxes, and synthetic waxes, such as, for example, polyalkylene waxes and polyethylene glycol waxes. In addition to the fats, suitable additives are also fat-like substances, such as lecithins and phospholipids. The term lecithins is understood by the person skilled in the art as meaning those glycerophospholipids which are founded from fatty acids, glycerol, phosphoric acid and choline by esterification. Lecithins are thus also often as phosphatidylcholines (PC) in the specialist world. Examples of natural lecithins which may be mentioned are the cephalins, which are also referred to as phosphatidic acids and constitute derivatives of 1,2-diacyl-sn-glycerol-3-phosphoric acids. By contrast, phospholipids are usually understood as meaning mono- and preferably diesters of phosphoric acid with glycerol (glycerol phosphates), which are generally classed as fats. In addition, sphingosines or sphingolipids are also suitable.

### Pearl Gloss Waxes

Suitable pearl gloss waxes include, for example: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which have a total of at least 24 carbon atoms, specifically laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

### Consistency Regulators and Thickening Agents

Suitable consistency regulators are primarily fatty alcohols or hydroxy fatty alcohols having 12 to 22, and preferably 16 to 18, carbon atoms, and also partial glycerides, fatty acids or hydroxy fatty acids. Preference is given to a combination of these substances with alkyl oligo-glucosides and/or fatty acid N-methylglucamides of identical chain length and/or polyglycerol poly-12-hydroxystearates. Suitable thickeners are, for example, Aerosil grades (hydrophilic silicas), polysaccharides, in particular xanthan gum, guar guar, agar agar, alginates and tyloses, carboxymethylcellulose, hydroxyethylcellulose and hydroxypropylcellulose, and also relatively high molecular weight polyethylene glycol mono- and diesters of fatty acids, polyacrylates (e.g. Carbopols®, and Pemulen grades from Goodrich; Synthalens® from Sigma; Keltrol grades from Kelco; Sepigel grades from Seppic; Salcare grades from Allied Colloids), polyacrylamides, polymers, polyvinyl alcohol and polyvinylpyrrolidone. Bentonites, such as, for example, Bentone®Gel VS 5PC (Rheox), which is a mixture of cyclopentasiloxane, disteardimonium hectorite and propylene carbonate, have also proven to be particularly effective. Also suitable are surfactants, such as, for example, ethoxylated fatty acid glycerides, esters of fatty acids with polyols such as, for example, pentaerythritol or trimethylolpropane, fatty alcohol ethoxylates having a narrowed homologue distribution or alkyl oligoglucosides, and electrolytes such as sodium chloride and ammonium chloride.

### Super-Fatty Agents

It is possible to use, as super-fatty solutions, substances such as, for example, lanoline and lecithin, as well as derivatives of lecithin and lanoline which are acylated or polyethoxylated, fatty acid esters of polyol, monoglycerides and alcanolamides of fatty acids, the latter also serving as foaming stabilizers.

### Stabilizers

It is possible to use, as stabilizers, metal salts of fatty acids, magnesium stearate, aluminum stearate and/or zinc stearate, or ricinoleate.

### Polymers

Suitable cationic polymers are, for example, cationic cellulose derivatives, such as, for example, a quaternized hydroxyethylcellulose obtainable under the name Polymer JR 400® from Amerchol, cationic starch, copolymers of diallylammonium salts and acrylamides, quaternized vinylpyrrolidone-vinylimidazole polymers, such as, for example, Luviquat® (BASF), condensation products of polyglycols and amines, quaternized collagen polypeptides, such as, for example, lauryldimonium hydroxypropyl hydrolyzed collagen (Lamequat®L/Grünau), quaternized wheat polypeptides, polyethyleneimine, cationic silicone polymers, such as, for example, amodimethicones, copolymers of adipic acid and dimethylaminohydroxypropyldiethylenetriamine (Cartaretins®/Sandoz), copolymers of acrylic acid with dimethyldiallylammonium chloride (Merquat® 550/Chemviron), polyaminopolyamides, as described, for example, in FR 2252840 A. and crosslinked water-soluble polymers thereof, cationic chitin derivatives, such as, for example, quaternized chitosan, optionally in microcrystalline dispersion, condensation products from dihaloalkyls, such as, for example, dibromobutane with bisdialkylamines, such as, for example, bis-dimethylamino-1,3-propane, cationic guar gum, such as, for example. Jaguar® CBS, Jaguar® C-17. Jaguar® C-16 from Celanese, quaternized ammonium salt polymers, such as, for example. Mirapol® A-15, Mirapol® AD-1, Mirapol® AZ-1 from Miranol.

Suitable anionic, zwitterionic, amphoteric and nonionic polymers are, for example, vinyl acetate-crotonic acid copolymers, vinylpyrrolidone-vinyl acrylate copolymers, vinyl acetate-butyl maleate-isobornyl acrylate copolymers, methyl vinyl ether-maleic anhydride copolymers and esters thereof, uncrosslinked polyacrylic acids and polyacrylic acids crosslinked with polyols, acrylamidopropyltrimethylammonium chloride-acrylate copolymers, octylacrylamide-methyl methacrylate-tert-butylaminoethyl methacrylate-2-hydroxypropyl methacrylate copolymers, polyvinylpyrrolidone, vinylpyrrolidone-vinyl acetate copolymers, vinylpyrrolidone-dimethylaminoethyl methacrylate-vinylcaprolactam terpolymers, and optionally derivatized cellulose ethers and silicones.

### Silicone-Containing Compounds

Suitable silicone compounds are, for example, dimethylpolysiloxanes, methylphenylpolysiloxanes, cyclic silicones, and amino-, fatty acid-, alcohol-, polyether-, epoxy-, fluorine-, glycoside- and/or alkyl-modified silicone compounds, which can either be liquid or in resin form at room temperature. Also suitable are simethicones, which are mixtures of dimethicones having an average chain length of from 200 to 300 dimethyl-siloxane units and hydrogenated silicates.

### Protective Filter Against UV Rays

UV photoprotective factors are, for example, to be understood as meaning organic substances (photoprotective filters) which are liquid or crystalline at room temperature and which are able to absorb ultraviolet rays and give off the absorbed energy again in the form of longer-wavelength radiation, e.g. heat. UVB filters can be oil-soluble or water-soluble. Examples of oil-soluble substances are:
3-benzylidenecamphor or 3-benzylidenenorcamphor and derivatives thereof, e.g. 3-(4-methylbenzylidene)camphor;
4-aminobenzoic acid derivatives, preferably 2-ethylhexyl 4-(dimethylamino)benzoate, 2-octyl 4-(dimethylamino)benzoate and amyl 4-(dimethylamino)benzoate;
esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenyl-cinnamate (octocrylene);
esters of salicylic acid, preferably 2-ethylhexyl salicylate, 4-isopropylbenzyl salicylate, homomethyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di-2-ethylhexyl 4-methoxybenzalmalonate;
triazine derivatives, such as, for example, 2,4,6-trianilino(p-carbo-2′-ethyl-1′-hexyloxy)-1,3,5-triazine and octyltriazone or dioctylbutamidotriazone (Uvasorb® HEB);
propane-1,3-diones, such as, for example, 1-(4-tert-butylphenyl)-3-(4′-methoxyphenyl)propane-1,3-dione;
ketotricyclo(5.2.1.0)decane derivatives.

Suitable water-soluble substances are:
2-phenylbenzimidazole-5-sulphonic acid and the alkali metal, alkaline earth metal, ammonium, alkylammonium, alkanolammonium and glucammonium salts thereof:
sulphonic acid derivatives of benzophenones, preferably 2-hydroxy-4-methoxybenzophenone-5-sulphonic acid and its salts;
sulphonic acid derivatives of 3-benzylidenecamphor, such as, for example, 4-(2-oxo-3-bornylidenemethyl)benzenesulphonic acid and 2-methyl-5-(2-oxo-3-bornyl-idene)sulphonic acid and salts thereof.

Suitable typical UV-A filters are include derivatives of benzylmethane, such as, for example, 1-(4′-tert-butylphenyl)-3-(4′-methoxyphenyl)propane-1,3-dione, 4-tert-butyl-4′-methoxydibenzoylmethane (Parsol® 1789), 1-phenyl-3-(4′-isopropylphenyl)propane-1,3-dione, and enamine compounds. The UV-A and UV-B filters can also be used in mixtures. Other suitable combinations include the derivatives of benzoylmethane, e.g. 4-tert-butyl-4′-methoxydibenzoylmethane (Parsol® 1789) and 2-ethylhexyl 2-cyano-3,3-phenylcinnamate (octocrylene) in combination with esters of cinnamic acid such as 2-ethylhexyl 4-methoxycinnamate and/or propyl 4-methoxycinnamate and/or isoamyl 4-methoxycinnamate. Such combinations may be combined with water-soluble filters such as, for example, 2-phenylbenzimidazole-5-sulphonic acid and their alkali metal, alkaline earth metal, ammonium, alkylammonium, alkanolammonium and glucammonium salts.

As well as said soluble substances, insoluble light protection pigments, namely finely dispersed metal oxides or salts, are also suitable for this purpose. Examples of suitable metal oxides include zinc oxide, titanium dioxide and also oxides of iron, zirconium, silicon, manganese, aluminium and cerium, and mixtures thereof. Salts which may be used are silicates (talc), barium sulphate or zinc stearate. The oxides and salts may be used in the form of the pigments for skincare and skin-protective emulsions and decorative cosmetics. The particles here may have an average diameter of less than 100 nm, between 5 and 50 nm, or between 15 and 30 nm. They can have a spherical shape, but it is also possible to use particles which have an ellipsoidal shape or a shape deviating in some other way from the spherical form. The pigments can also be surface-treated, i.e. hydrophilicized or hydrophobicized. Typical examples include coated titanium dioxides, such as, for example, titanium dioxide T 805 (Degussa) or Eusolex®-T2000 (Merck). Suitable hydrophobic coating agents include silicones, trialkoxyoctylsilanes or simethicones. In sunscreens, micro- or nanopigments may be used such as micronized zinc oxide.

### Biogenic and Antioxidant Substances

Biogenic substances should be understood to refer to, for example, tocopherol, tocopherol acetate, tocopherol palmitate, ascorbic acid, (deoxy)ribonucleic acid and the fragmentation products thereof, β-glucans, retinol, bisabol, allantoine, phytantriol, panthenol, AHA acids, amino acids, ceramides, pseudoceramides, essential oils, plant extracts, as well as, for example, prunus extract, bambaranus extract and vitamin-containing complexes.

Antioxidants interrupt the photochemical reaction chain which is triggered when UV radiation penetrates the skin. Typical examples include carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodi-propionate, thiodipropionic acid and derivatives thereof (esters, ethers, peptides, lipids, nucleotides, nucleosides and salts), and sulphoximine compounds (e.g. buthionine sulphoximines, homocysteine sulphoximine, buthionine sulphones, penta-, hexa-, heptathionine sulphoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of gum benzoin, rutic acid and derivatives thereof, α-glycosylrutin, ferulic acid, furfurylideneglucitol, camosine, butylhydroxytoluene, butylhydroxyanisole, nordihydro-guaiacic acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO4) selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

### Deodorants and Substances Inhibiting Germs

Cosmetic deodorants (deodorising agents) counteract, mask or remove body odors. Body odors arise as a result of the effect of skin bacteria on apocrine perspiration, with the formation of degradation products which have an unpleasant odor. Accordingly, deodorants comprise active ingredients which act as antimicrobial agents, enzyme inhibitors, odor absorbers or odor masking agents.

### Antimicrobial Agents

Suitable antimicrobial agents are, in principle, all substances effective against gram-positive bacteria, such as, for example, 4-hydroxybenzoic acid and its salts and esters, N-(4-chlorophenyl)-N′-(3,4-dichlorophenyl)urea, 2,4,4′-trichloro-2′-hydroxydiphenyl ether (triclosan), 4-chloro-3,5-dimethylphenol, 2,2′-methylenebis(6-bromo-4-chlorophenol), 3-methyl-4-(1)-methylethyl)phenol, 2-benzyl-4-chlorophenol, 3-(4-chlorophenoxy)-1,2-propanediol, 3-iodo-2-propynyl butylcarbamate, chlorhexidine, 3,4,4′-trichlorocarbanilide (TTC), antibacterial fragrances, thymol, thyme oil, eugenol, oil of cloves, menthol, mint oil, farnesol, phenoxyethanol, glycerol monocaprate, glycerol monocaprylate, glycerol monolaurate (GML), diglycerol monocaprate (DMC), salicylic acid N-alkylamides, such as, for example, N-octylsalicylamide or N-decylsalicylamide.

### Enzyme Inhibitors

Suitable enzyme inhibitors are, for example, esterase inhibitors. These include trialkyl citrates, such as trimethyl citrate, tripropyl citrate, triisopropyl citrate, tributyl citrate, triethyl citrate (Hydagen® CAT). The substances inhibit enzyme activity, thereby reducing the formation of odor. Other substances which are suitable esterase inhibitors are sterol sulphates or phosphates, such as, for example, lanosterol, cholesterol, campesterol, stigmasterol and sitosterol sulphate or phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

### Odor Absorbing Agents

Suitable odor absorbers are substances which are able to absorb and largely retain odor-forming compounds. They lower the partial pressure of the individual components, thus also reducing their rate of diffusion. It is important that in this process perfumes must remain unimpaired. Odor absorbers are not effective against bacteria. They comprise, for example, as main constituent, a complex zinc salt of ricinoleic acid or specific, largely odor-neutral fragrances which are known to the person skilled in the art as “fixatives”, such as, for example, extracts of labdanum or styrax or certain abietic acid derivatives. The odor masking agents are fragrances or perfume oils, which, in addition to their function as odor masking agents, give the deodorants their respective fragrance note. Perfume oils which may be mentioned are, for example, mixtures of natural and synthetic fragrances. Natural fragrances are extracts from flowers, stems and leaves, fruits, fruit peels, roots, woods, herbs and grasses, needles and branches, and resins and balsams. Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate. p-tert-butylcyclohexyl acetate, linalyl acetate, phenylethyl acetate, linalyl benzoate, benzyl formate, allyl cyclohexylpropionate, styrallyl propionate and benzyl sali-cylate. The ethers include, for example, benzyl ethyl ether, and the aldehydes include, for example, the linear alkanals having 8 to 18 carbon atoms, citral, citronellal, citronellyloxyacetaldehyde, cyclamen aldehyde, hydroxycitronellal, lilial and bourgeonal, the ketones include, for example, the ionones and methyl cedryl ketone, the alcohols include anethole, citronellol, eugenol, isocugenol, geraniol, linalool, phenylethyl alcohol and terpineol, and the hydrocarbons include mainly the terpenes and balsams. Preference is, however, given to using mixtures of different fragrances which together produce a pleasing fragrance note. Essential oils of relatively low volatility, which are mostly used as aroma components, are also suitable as perfume oils, e.g. sage oil, camomile oil, oil of cloves, melissa oil, mint oil, cinnamon leaf oil, linden blossom oil, juniper berry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate. Vertofix coeur, iso-E-super, Fixolide NP, evemyl, iraldein gamma, phenylacetic acid, geranyl acetate, benzyl acetate, rose oxide, romilat, irotyl and floramat alone or in mixtures.

### Anti-Perspirants

Antiperspirants reduce the formation of perspiration by influencing the activity of the eccrine sweat glands, thus counteracting underarm wetness and body odor. Aqueous or anhydrous formulations of antiperspirants typically comprise the following ingredients:
astringent active ingredients,
oil components,
nonionic emulsifiers,
coemulsifiers,
consistency regulators,
auxiliaries, such as, for example, thickeners or complexing agents and/or
nonaqueous solvents, such as, for example, ethanol, propylene glycol and/or glycerol.

Suitable astringent antiperspirant active ingredients are primarily salts of aluminium, zirconium or of zinc. Such suitable antihydrotic active ingredients are, for example, aluminium chloride, aluminium chlorohydrate, aluminium dichlorohydrate, aluminium sesquichlorohydrate and complex compounds thereof, e.g. with 1,2-propylene glycol, aluminium hydroxyallantoinate, aluminium chloride tartrate, aluminium zirconium trichlorohydrate, aluminium zirconium tetrachlorohydrate, aluminium zirconium penta-chlorohydrate and complex compounds thereof, e.g. with amino acids, such as glycine. In addition, customary oil-soluble and water-soluble auxiliaries may be present in antiperspirants in relatively small amounts. Such oil-soluble auxiliaries may, for example, be:
anti-inflammatory, skin-protective or perfumed essential oils,
synthetic skin-protective active ingredients and/or
oil-soluble perfume oils.

Customary water-soluble additives are, for example, preservatives, water-soluble fragrances, pH regulators, e.g. buffer mixtures, water-soluble thickeners, e.g. water-soluble natural or synthetic polymers, such as, for example, xanthan gum, hydroxyethylcellulose, polyvinylpyrrolidone or high molecular weight polyethylene oxides.

### Film-Forming Agents

Conventional film-forming agents include quitosane, microcrystalline quitosane, quaternised quitosane, polyvinyl pyrrolidone, copolymerisates of vinyl pyrrolidone/vinyl acetate, polymers from the series of acrylic acids, quaternary cellulose derivatives, collagen, hyaluric acid, or the salts thereof and similar compounds.

### Expansion Agents

Aqueous phase expansion agents may include montmorillonite, mineral substances of pemulen clay, as well as types of carbopol modified by the alkyl (Goodrich).

### Insect Repelling Agents

Suitable insect repellents include N,N-diethyl-m-toluamide, 1,2-pentanediol and ethyl butylacetylaminopropionate.

### Self-Tanning Agents and Depigmentation Agents

Dihydroxyacetone is suitable as a self-tanning agent. Inhibitors of tyrosine which prevent the formation of melanin and which are used in depigmentation solutions may include arbutin, ferulic acid, kojic acid, coumarin acid and ascorbic acid (vitamin C).

### Hydrotropes

To improve the flow behaviour, it is also possible to use hydrotropic agents, such as, for example, ethanol, isopropyl alcohol, or polyols. Polyols which are suitable here include polyols with 2 to 15 carbon atoms and at least two hydroxyl groups. The polyols can also contain further functional groups, in particular amino groups, or be modified with nitrogen. Typical examples are:
glycerol;
alkylene glycols, such as, for example, ethylene glycol, diethylene glycol, propylene glycol, butylene glycol, hexylene glycol, and polyethylene glycols with an average molecular weight of from 100 to 1 000 daltons;
technical-grade oligoglycerol mixtures with a degree of self-condensation of from 1.5 to 10, such as, for example, technical-grade diglycerol mixtures with a diglycerol content of from 40 to 50% by weight;
methylol compounds, such as, in particular, trimethylolethane, trimethylolpropane, trimethylolbutane, pentaerythritol and dipentaerythritol;
lower alkyl glucosides, in particular those having 1 to 8 carbon atoms in the alkyl radical, such as, for example, methyl and butyl glucoside;
sugar alcohols having 5 to 12 carbon atoms, such as, for example, sorbitol or mannitol,
sugars having 5 to 12 carbon atoms, such as, for example, glucose or sucrose;
amino sugars, such as, for example, glucamine;
dialcohol amines, such as diethanolamine or 2-amino-1,3-propanediol.

### Preservative Agents

Suitable preservative agents include, for example, phenoxyethanol, a solution of formaldehyde, paraben, pentanediol and sorbic acid, as well as silver complexes which are known under the commercial reference Surfacine® and other classes of substances set out in annex 6, parts A and B of the cosmetic regulations.

### Perfume and Aromatic Oils

Perfume oils which may be mentioned are mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamon, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl-methylphenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamen aldehyde, hydroxycitronellal, lilial and bourgeonal, and the ketones include, for example, the ionones, α-isomethylionone and methyl cedryl ketone, the alcohols include anethole, citronellol, eugenol, isocugenol, geraniol, linalool, phenylethyl alcohol and terpineol, and the hydrocarbons include mainly the terpenes and balsams. Essential oils of relatively low volatility, which are mostly used as aroma components, are also suitable as perfume oils, e.g. sage oil, camomile oil, oil of cloves, melissa oil, mint oil, cinnamon leaf oil, linden blossom oil, juniper berry oil, vetiver oil, olibanum oil, galbanum oil, labolanum oil and lavandin oil. Other suitable oils include bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

Suitable aromas are, for example, peppermint oil, spearmint oil, anise oil, star anise oil, caraway oil, eucalyptus oil, fennel oil, lemon oil, wintergreen oil, oil of cloves, menthol and the like.

### Methods of Treatment

In some embodiments, the present invention is directed to a method of treating forehead wrinkles comprising topically administering a cosmetic composition according to an embodiment to the forehead of a subject in need thereof.

In some embodiments, the present invention is directed to a method of treating nasal folds comprising topically administering a cosmetic composition according to an embodiment to the affected area of a subject in need thereof.

In some embodiments, the present invention is directed to a method of treating crow's feet comprising topically administering a cosmetic composition according to an embodiment to the affected area of a subject in need thereof.

In some embodiments, the present invention is directed to a method of increasing cheek density comprising topically administering a cosmetic composition according to an embodiment to the cheek of a subject in need thereof.

In some embodiments, the present invention is directed to a method of improving hypoechogenic dermal factor comprising topically administering a cosmetic composition according to an embodiment to the affected area of a subject in need thereof.

Methods according to embodiments for treating one or more of forehead wrinkles, nasal fold, crow's feet, cheek density, or hypoechogenic dermal factor comprise administering a cosmetic composition according to an embodiment for at least 7 days, at least 10 days, at least 14 days, at least 21 days, at least 42 days, or at least 56 days.

### ILLUSTRATIVE EXAMPLES

### Example 1: Synergistic Blend Study

Key inclusion Criteria:
45-65 years old, female
Fitzpatrick skin type I, II, or III
Have Crow's Feet wrinkles of grade 3-4, nasolabial fold of grade 2-3 and forehead wrinkles assessed by a scientist
Consider themselves to have loss of skin elasticity and firmness

Application
On face, twice a day for 56 days

Measurements
Wrinkles ranking on Visia Images
Cheek dermal density and hypoechogenic dermal factor by ultrasounds

Cosmeic Composition

| | Weight |
|---|---|
| Ingredient | Percent |
| Water | 78.97 |
| Mannitol (and) Acetyl Tetrapeptide-9 | 1.00 |
| Mannitol (and) Acetyl Tetrapeptide-11 | 1.00 |
| Dimethyl Isosorbide | 2.00 |
| Sodium Stearoyl Glutamate | 0.08 |
| Glycerin | 2.00 |
| Xanthan Gum | 0.10 |
| Sodium Polyacrylate | 1.50 |
| Sodium Lauryl Glucose Carboxylate (and) | 1.75 |
| Lauryl Glucoside | |
| Polyglyceryl-2 Dipolyhydroxystearate | 2.00 |
| Coco-caprylate | 6.50 |
| Polysorbate 20 | 2.00 |
| Phenoxyethanol (and) Ethylhexylglycerin | 1.10 |

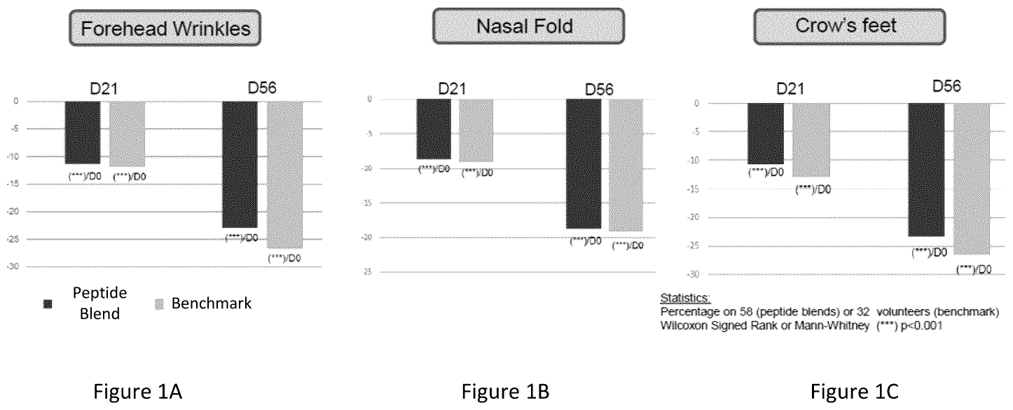

FIG. 1A-1C illustrate the improvement in forehead wrinkles, nasal fold and Crow's feet, respectively, after as early as 21 days for the peptide combination as compared to a benchmark product. The peptide combination performs as good as the benchmark. Efficacy of the peptide combination further improves over time with at least a two time better improvement after 56 days.

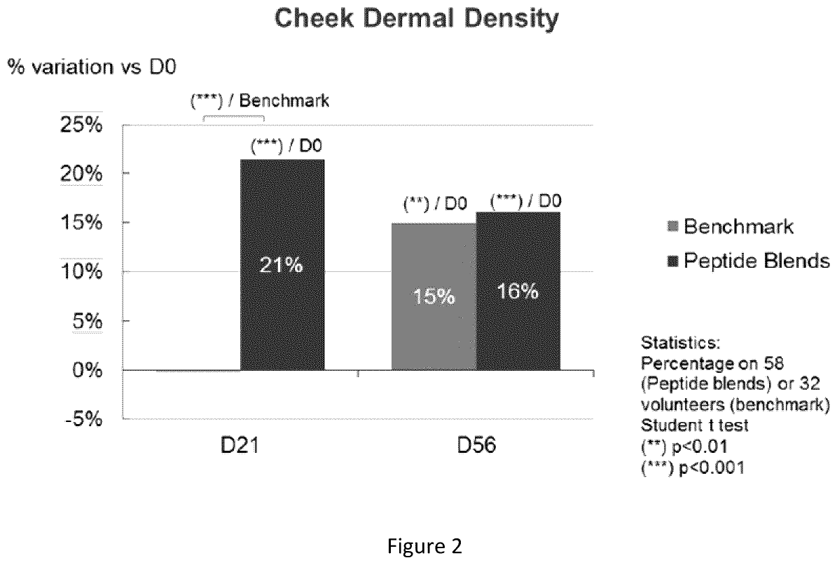

FIG. 2 illustrates the improvement in cheek dermal density, as measured by ultrasound, after as early as 21 days for the peptide combination as compared to a benchmark product. After 21 days, there is a 21% improvement in the cheek dermal density with the peptide combination. In contrast, no dermal density improvement was observed with the benchmark product after 21 days. Efficacy of the peptide combination is confirmed after 56 days.

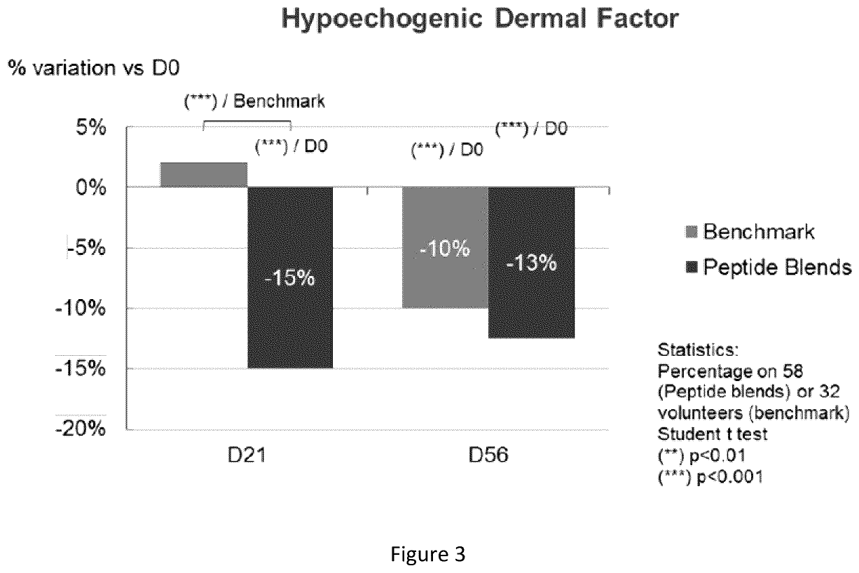

FIG. 3 illustrates the improvement in cheek hypoechogenic dermal factor, as measured by ultrasound, after as early as 21 days for the peptide combination as compared to a benchmark product. After 21 days, there is a 15% improvement in the cheek hypoechogenic dermal factor. In contrast, no cheek hypoechogenic dermal factor improvement was observed with the benchmark product after 21 days. Efficacy of the peptide combination is confirmed after 56 days. In light of the above results, it has been shown that the peptide combination provides for a quicker firming effect when compared to the benchmark product.

Furthermore, the peptide combination results in a synergistic effect on the improvement of one or more of forehead wrinkles, nasal fold, crow's feet, cheek density, or hypoechogenic dermal factor. In clinical studies with 3% Dermican™ (comprising SEQ ID NO:1), 4 months of bi-daily application were needed to achieve an improvement of skin firmness, whereas no improvement was observed on skin firmness after the first 2 months of application. In clinical studies with 3% Syniorage™ (comprising SEQ ID NO:2), 2 months of bi-daily application were needed to improve the skin texture, skin smoothing, and the biomecanbical properties of firmness and elasticity. In comparison to the performance of the individual peptides, the peptide combination illustrated improvements in skin firmness in as early as 21 days.

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

Reference throughout this specification to numerical ranges should not be construed as limiting and should be understood as encompassing the outer limits of the range as well as each number and/or narrower range within the enumerated numerical range.

The term “about”, when referring to a physical quantity, is to be understood to include measurement errors within, and inclusive of 10%. For example, “about 100° C.” should be understood to mean “100±10° C.”.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A cosmetic composition comprising a synergistic combination of (i) N-Acetyl-Glutaminyl-Aspartyl-Valyl-Histidine (SEQ ID NO:1), and (ii) N-Acetyl-Prolyl-Prolyl-Tyrosyl-Leucine (SEQ ID NO:2).

2. (canceled)

3. The cosmetic composition of claim 1, further comprising at least one cosmetically acceptable excipient.

4.-13. (canceled)

14. The cosmetic composition of claim 1, comprising a molar ratio of SEQ ID NO:1 to SEQ ID NO:2 of about 1.3:1 to about 1:2.3.

15. The cosmetic composition of claim 1, comprising about 250 to about 1300 ppm of SEQ ID NO:1.

16. The cosmetic composition of claim 1, comprising about 350 to about 1650 ppm of SEQ ID NO:2.

17. (canceled)

18. The cosmetic composition of claim 1, wherein the composition exhibits an improvement in one or more of forehead wrinkles, nasal fold, crow's feet, cheek density, or hypoechogenic dermal factor after a 21 day treatment.

19. A method of treating forehead wrinkles comprising topically administering a cosmetic composition to the forehead of a subject in need thereof, wherein the cosmetic composition comprises a synergistic combination of (i) N-Acetyl-Glutaminyl-Aspartyl-Valyl-Histidine (SEO ID NO:1), and (ii) N-Acetyl-Prolyl-Prolyl-Tyrosyl-Leucine (SEO ID NO:2.

20. A method of treating nasal folds comprising topically administering a cosmetic composition to the affected area of a subject in need thereof, wherein the cosmetic composition comprises a synergistic combination of (i) N-Acetyl-Glutaminyl-Aspartyl-Valyl-Histidine (SEO ID NO:1), and (ii) N-Acetyl-Prolyl-Prolyl-Tyrosyl-Leucine (SEO ID NO:2.

21. A method of treating crow's feet comprising topically administering a cosmetic composition of claim 1 to the affected area of a subject in need thereof.

22. A method of increasing cheek density comprising topically administering a cosmetic composition of claim 1 to the cheek of a subject in need thereof.

23. A method of improving hypoechogenic dermal factor comprising topically administering a cosmetic composition of claim 1 to the affected area of a subject in need thereof.

24. The method of claim 19, wherein the administration is for at least 7 days, at least 10 days, at least 14 days, at least 21 days, at least 42 days, or at least 56 days.


SEQUENCE LISTING (2 sequences)

SEQ ID NO: 1 - protein, 4 aa, synthetic construct
  REGION 1..4: note = Description of Artificial Sequence: Synthetic peptide
  REGION 1..4: note = N-term Acetyl
QDVH

SEQ ID NO: 2 - protein, 4 aa, synthetic construct
  REGION 1..4: note = Description of Artificial Sequence: Synthetic peptide
  REGION 1..4: note = N-term Acetyl
PPYL